(19) United States
(12) Patent Application Publication
(10) Pub. No.: US 2019/0321273 A1
(43) Pub. Date: Oct. 24, 2019

(54) USE OF CERTAIN COMPOUNDS TO MODIFY, REDUCE, OR ELIMINATE OFF-NOTES

(71) Applicant: SYMRISE AG, Holzminden (DE)

(72) Inventors: Nicole SCHULZE, Freden (Leine) (DE); Celina PETERS, Bad Pyrmont (DE)

(21) Appl. No.: 16/310,289

(22) PCT Filed: Jun. 21, 2016
(86) PCT No.: PCT/EP2016/064269

§ 371 Date: Dec. 14, 2018

(51) Int. Cl. A61K 8/22; A61K 8/42; A61Q 13/00; A23L 29/00

(52) U.S. Cl. CPC A61K 8/22; A61K 8/42; A61K 2800/81; A23L 29/015; A23L 29/045; A61Q 13/00

(57) ABSTRACT

The present invention primarily concerns the use of one or more compound(s) with the Formula (I)

XₖYₗZₘOₙ (I),

as described herein, to modify, reduce or eliminate one or more off-note(s) of one or more substance(s) selected from the group (G1) as described herein, through chemical conversion of the substance or rather the substances of the group (G1) and thereupon modifying, reducing or eliminating the off-note(s), in a composition (Z1), which next to one or more compounds of the Formula (I) and the substance or rather the substances of the group (G1) further contains one or more substances selected from the group (G2) as described herein. Furthermore, the present invention concerns new methods for modifying, reducing or eliminating one or more off-note(s) and new compositions obtained therefrom.

The present invention primarily concerns the use of one or more compound(s) with the Formula (I) (as described herein) to modify, reduce or eliminate one or more off-note(s) of one or more substance(s) selected from the group (G1) as described herein, by chemical conversion of the substance or rather the substances of the group (G1) and thereby modifying, reducing or eliminating of the off-note(s), in a composition (Z1), which next to one or more compounds of the Formula (I) and the substance or rather the substances of the group (G1) further contains one or more substances selected from the group (G2), as described herein. Further, the present invention concerns new methods for modifying, reducing or eliminating one or more off-note(s) and new, compositions obtained therefrom. Further aspects and preferred embodiments of the present invention result from the following description as well as particularly from the attached patent claims.

The compounds of the Formula (I) described herein are advantageously suited for the pre-treatment for the improvement of the sensory properties of unperfumed or non-aromatised basic substances or compositions, are used for the production of perfumed or aromatised products for the sectors of cosmetics, personal care, oral hygiene products, oral care, household, air fresheners, fine fragrance, perfumes, foodstuff, food supplements, flavours, pet food, pharmaceutical products.

Unperfumed or non-aromatised basic substances/ingredients and compositions/formulations/preparations prepared therefrom industrially often have an intrinsic odour or an intrinsic aroma, which is undesirable in most of the cases and is referred to as off-note (off-aroma, off-note, bad odour, malodour, taste error, erroneous flavour or off-flavour). This off-note resulting from the basic substances/ingredients often influences the sensory properties and the profile of the perfumed or aromatised products/final products/preparations prepared therefrom. The sensory properties or rather the scent or the aroma of a product/final product/preparation are decisive quality features, which can be both decisive for purchase and decisive for the market, and an undesirable off-note would influence the quality of the perfumed or aromatised product/final product negatively. Thus, there is a constant interest to reduce or to eliminate the undesirable off-note and to thereby to improve the quality of the products.

A possible way towards the reduction of the undesirable off-note is the chemical modification or rather reaction, at which the compound(s) causing the off-notes interact and are converted with one or more agent(s), which leads to a reduction of the sensorially perceived impression of the undesirable off-note(s). In the following paragraphs, known procedures and methods are described, which use the principle of the chemical modification.

In U.S. Pat. No. 5,789,010, a method for the reduction of undesirable aldehyde-compounds in fats or rather triglycerides or their derivatives by the addition of a Schiff base is described, in which the undesirable aldehyde-compound is bound and thereby a new desirable aldehyde-compound is released. The disadvantage of the method is, that the released new aldehydes can influence the fragrance of the desired perfume. Furthermore, Schiff bases are often coloured and can lead to undesirable discolouring of the final product.

GB 1596752 describes a chemical method for the reduction of undesirable carboxylic acids by alcoholamines, wherein these form non-volatile complexes with the undesirable carboxylic acids and thereby reduce the intensity of the off-note, which is caused by the carboxylic acids.

The reduction or rather removal of the off-note of H₂S in cresol-soap-solution by the chemical conversion of the H₂S with H₂O₂ to elementary sulphur as well is an already known method (CN1100259A).

By the use of acids, the release of aminous off-notes can be reduced or avoided, as described in WO2006131739.

Sulphur-containing and nitrogen-containing malodours can be chemically converted and neutralised by a complex from dialkimonometal and an organic, polyfunctional chelate ligand in presence of oxygen, which releases active metal ions in aqueous, buffered medium (U.S. Pat. No. 5,534,249 (A)). Thus, malodours of carpet cleaners, in container toilets or of ammonia can be neutralised.

Olefinic compounds and mixtures, which are contaminated with sulphur-containing malodours, of exemplarily terpentine, cyclohexene, dipentene, pine oil and anethol can be deodorised by the method described in U.S. Pat. No. 3,909,395 (A) by peralkanoic acids, such as peracetic acid, by oxidising the sulphur-containing malodours and post-treating by optionally washing steps, pH-value neutralisation or a steam distillation.

In the document KR 100447305 B1, a deodorant with malodour-reducing properties and without harmful effect for humans is described, wherein the malodours are removed by oxidation, reduction, neutralisation and absorption by a mixture of 6.7-6.8 wt.-% eucalyptus extract, 0.4-0.6 wt.-% stabilized chlorine dioxide, 0.2-0.3 wt. % zeolithe, 0.6-0.8 wt.-% aluminium sulfate, 0.04-0.05 wt.-% acetic acid and 89.54-93.97 wt.-% water.

The sulphur- and nitrogen-containing malodours can be fought by using dialkyl esters of fumaric acid, which react with the corresponding odorous molecules, wherein the reaction products have a hardly perceptible odour (DE 2335091A1).

The document U.S. Pat. No. 7,588,790 B2 describes a method for the reduction or removal of the undesirable, bitter off-note in potassium lactate by the use of activated carbon, whereby the activated carbon remains as residue or is separated or rather filtered off.

The reduction or removal of undesirable notes can occur via polymers, which prior to the application have been embossed to undesirable molecules, and then bind these (EP0925776A2). These polymers can be applied in diverse applications and products. In this method, the polymers remain as residue or are separated in a subsequent step.

A routinely applied method for the removal of undesirable off-notes is given by the distillation, optionally by the addition of further compounds and auxiliaries, such as bases or alkaline-earth metals (DE 180499; U.S. Pat. No. 2,801,209). Distillation steps for the processing require a lot of energy and large distillation apparatuses for the industrial production.

A further common solution approach is the covering or masking of the undesirable off-notes by the addition of fragrances and aromas as well as neutralizers or modulators, as they are exemplarily described in following documents: WO 2002085294, EP2133102; U.S. Pat. Nos. 5,559,271, 5,501,805, WO 0143784. Disadvantage of this method is, that the added fragrances and aromas for masking or rather covering influence the profile of the selected flavouring or perfuming and can lead to a modified profile and thus to modified sensory properties. The modifications are frequently not desired or rather accepted.

There is a constant need for possibilities to improve the quality and thus the sensory properties of the products and preparations in the market.

The primary task of the present invention was thus to develop a simple solution, with which the sensorially perceived intensity of the undesirable off-note(s) of certain substances and compositions can be modified, reduced or even eliminated entirely. Thereupon, the solution was intended to influence a (later) perfuming or flavouring as little as possible. The solution itself was intended to preferably not introduce any new/additional noteworthy sensory intensities and not merely mask or cover the off-note by the use of fragrances or aromas. Colour changes are also undesirable as they may lead to discolouration of the final product/preparation, which in turn may have a negative impact on quality or acceptance. Further tasks result from the following description as well as particularly the attached patent claims.

The primary task is solved by the use of one or more compounds of Formula (I)

XₖYₗZₘOₙ (I),

wherein X=N, Na, K, Mg or Ca, with k=0, 1 or 2, Y=C or S, with I=0, 1, 2 or 3, and Z=H, with m=0, 1, 2, 3, 4, 5, 6, 7 or 8, and n=2, 3, 4, 5, 6, 7 or 8, preferentially with Z=H, with m=0, 1, 2, 3, 4, 5 or 6, and n=2, 3, 4, 5 or 6,

to modify, reduce or eliminate one or more off-note(s) of one or more substance(s) selected from the group (G1) consisting of aldehydes, ketones, unsaturated hydrocarbons and amines, through chemical conversion of the substance or rather substances of the group (G1) and thereupon modifying, reducing or eliminating of the off-note(s), in a composition (Z1), which next to one or more compounds of Formula (I) and the substance or rather the substances of the group (G1) further contains one or more substances selected from the group (G2), consisting of alcohols, carboxylic acids, esters, carboxylates, mineral salts, saturated hydrocarbons, silicones, quaternary ammonium compounds, sulfates and sulphuric acid esters.

Chemical conversion of the substance or rather of the substances of the group (G1) is understood in the scope of the present invention preferably, that the substance(s) selected from the group (G1) is/are converted in (a) substance(s), which is/are less volatile and/or have a higher perception threshold and/or a more pleasant/desired odour or rather a more pleasant/desired aroma. Substances of the group (G1) are converted in the scope of the present invention preferentially in their alcohols, oxides, carboxylic acids, salts, esters or oligomers or rather polymers. In view of the Example section further below, here it is at this point exemplarily pointed to the compound trimethylamine (gaseous, melting point −117° C.; boiling point 2.9° C.; vapour pressure of 1887 hPa (20° C.); unpleasant fish smell), which in the scope of the present invention is preferentially converted to trimethylamine oxide (solid; melting point 213° C.; odourless). Next to the compounds of Formula (I) listed in the Example section, numerous further compounds of Formula (I) were analysed in the scope of comprehensive studies in view of their usability according to the invention with the result, that compounds of Formula (I), which are described herein as preferential, are especially suitable for the purposes of the present invention.

As compounds of Formula (I), which are described as preferential in the scope of the present invention, can particularly be understood such, which are according to the Formula (I′):

XₖYₗZₘOₙ (I′),

wherein X=N, Na or K, with k=0, 1 or 2, Y=C or S, with I=0 or 1, and Z=H, with m=1, 2 or 3, and n=2, 3, 5 or 6.

Particularly preferred compounds of the Formula (I) can exemplarily be selected from the group consisting of percarbamide, sodium percarbonate, KHSOS and hydrogen peroxide.

As off-note (off-aroma, off-note, bad odour, malodour, taste error, erroneous flavour or off-flavour) can in the scope of the present invention be understood any sensory (of taste and/or of smell, particularly unpleasant or undesirable of taste and/or of smell) impression, which a substance of the group (G1) (as described herein) has, be it as primary and/or as aftertaste and/or smell. Preferred off-notes to be modified, to be reduced or to be eliminated in the scope of the present invention are described further below.

The molecular weight of the one or rather one, more or all compound(s) of the Formula (I) is preferably in the range from 500 g/mol through 30 g/mol, further preferably in the range from 300 g/mol through 30 g/mol, especially preferentially in the range from 160 g/mol through 30 g/mol.

In the scope of a use according to the invention, the sensorially perceived intensity of the off-note is reduced preferably by 20% and more in comparison with the untreated sample or rather an identical composition without compounds of Formula (I) to be used according to the invention (see therefore also composition (Z0) as described herein further below).

The compound or the compounds of the Formula (I) are consumed during the use and preferably remain in the composition only in small amounts, wherein the amount of residue is preferably at 0.05 wt.-% and less, exemplarily between 0.00001 through 0.05 Gew-%, (see therefore also composition (Z2) as described herein further below).

An advantage of the present invention is, that this type of off-note modification, reduction or rather elimination is applicable for different off-note issues, as it is effective for a broad spectrum of undesirable off-notes and different substances causing these off-notes (see group (G1)).

Further advantageous about this invention is, that independently of the intensity of the undesirable off-note, partly even small amounts of compound(s) of the Formula (I) are sufficient. According to the invention it is preferred, when the total amount of compound(s) of Formula (I) is in the range from 0.001 through 30 wt.-%, preferably in the range from 0.01 through 20 wt.-%, especially preferentially in the range from 0.04 through 15 wt.-%, relative to the total weight of the composition (Z1).

The compositions obtained by or rather after use according to the invention (see therefore also composition (Z2) as described herein further below) can advantageously be processed further without further processing steps such as filtrations or distillations and are prepared or rather suitable for perfumery or rather flavouring. The solution approach according to the invention does advantageously not adversely affect the added, value-giving perfumes or aromas. Furthermore, the solution described herein is to be carried out simply and without large effort, comprises few steps and no additional steps of filtrations or of distillations; Further, it is energy saving and does not require complex or expensive apparatuses.

The used compounds of Formula (I) themselves preferably have no noteworthy odour or no noteworthy aroma, whereby these do not modify the sensory profile at a later perfuming or flavouring. The (further) processing of compositions with reduced off-note(s) enables advantageously the production of perfumed/aromatized products with improved sensory properties and thus an improved quality.

A further advantage in comparison with the mere masking or covering is, that this invention is advantageously a real reduction or even removal of the off-note(s), wherein the effect, depending on the type of the undesirable off-note, is measurable both sensorially and analytically (cf. Example section below).

Surprisingly it was shown, that the use of one or more compound(s) of Formula (I) in perfumed or aromatized products, a reduction of the intensity of the perceived off-note(s) at a simultaneous increase of the perceived intensity of the perfume or rather of the aromas could be found.

The total amount of substance(s) of the group (G1) is preferably at 0.1 wt.-% or less, preferentially at 0.01 wt.-% or less, especially preferentially at 0.001 wt.-% or less, particularly preferentially at 0.0001 wt.-% or less, relative to the total weight der composition (Z1) or the headspace of the composition at 25° C. and atmospheric pressure.

In view of the substance or rather the substances causing the off-note(s), it preferably holds, that the substance or rather the or one, more or all substances of the group (G1) is or rather are each selected independently from each other selected from the group (G1′), consisting of linear, branched, cyclic or rather aromatic aldehydes, preferably of such with 1 through 20 carbon atoms, linear, branched, cyclic or rather aromatic ketones, preferably of such with 3 through 20 carbon atoms, linear or branched unsaturated hydrocarbons, preferably of such with 2 through 20 carbon atoms and linear, branched or cyclic amines, preferably of such with 1 through 20 carbon atoms, and/or of the group (G1″), consisting of compounds with a molecular mass from 18 through 700 g/mol, preferentially 18 through 500 g/mol, especially preferentially 18 through 300 g/mol, particularly preferentially 30 through 300 g/mol, preferably selected from the group (G1′″), consisting of formaldehyde, acetaldehyde, propanal, butanal, pentanal, hexanal, heptanal, octanal, nonanal, decanal, undecanal, dodecanal, tetradecanal, hexadecanal, octadecanal, decadienal, dodecandienal, heptadienal, hexadienal, octadienal, undecandienal, decenal, undecenal, heptenal, trienales and further, octenone, octenal, nonenal, decenal, propanone, butanone, pentanone, pentenone, octadienone, methylnonadienone, pentylfuran, hexanone, heptanone, decanone, methylheptenone, methylheptadienone, methylpropanal, methylbutanal, phenylethanal, hydroxymethylfuranone, hydroxydimethylfuranone, hydroxyethylfuranon, acrolein, benzaldehyde, furfural, furylidenketone, carene, indene, squalene, ocimene, limonene, terpinene, phellandrene, pinene, myrcene, tridecene, tetradecene, dodecene, pentadecene, undecene, decene, nonene, octadecene, heptadecene, hexadecene, farnesene, humulene, butene, pentene, methylpentene, butandiene, methylbutadiene, undectriene, longifolene, menthadiene, methylbutene, styrene, methylamine, dimethylamine, trimethylamine, ethylamine, diethylamine, triethylamine, butylamine, ethanolamine, diethanolamine, triethanolamine and furfurylamine.

A desirable off-note is frequently described with terms such as exemplarily roasted, nutty, spicy, bitter, stinging, biting, scratchy, medicinal, fishy, aminous, hay-like, chemical, technical, burnt, fatty, sour, herbaceous, musty, rotten, fermented, terpenous, aromatic, fruity, woody, fleshy, ethereal, earthy, green, solvent-like, rubbery, charred, faecal, urinary, earthy, mushroomy, smoky, decaying, rancid, gasoline-like, buttery, vegetable-like, cooked, animalistic, sweaty, cheesy, cabbage-like, hot, cooling, creamy, flowery, ointment-like, mossy, metallic, workshop-like, indoor swimming pool-like, pyridine-like, pyrazine-like, ensilage-like, carrot-like, adhesive-like, disinfectant-like, dusty, spicy, aromatic, powdery, papery, floury, dry. Such off-notes are in the scope of the present invention especially preferentially off-notes to be modified, reduced or eliminated.

The mass ratio of the total amount of compound(s) of Formula (I) to the total amount of substance(s) of the group (G1) in the composition (Z1) is preferably in the range from 300:1 through 1:3, preferably in the range from 100:1 through 1:3, especially preferentially in the range from 10:1 through 1:2, especially preferentially in the range from 3:1 through 1:1.

Substances of the group (G2) are preferably—at least in part—common unperfumed or non-aromatized basic compounds/ingredients as they are applied in the production of perfumed or aromatized products, compositions and preparations from the sectors of personal care, oral care, household, fine fragrance, foodstuff, pet food, flavour, pharmacy.

It is preferred according to the invention, when the substance or the or rather one, more or all of the substances of the group (G2) contained in the composition (Z1) is or rather are selected from the group (G2′) consisting of compounds with a molecular mass in the range from 18 through 2000 g/mol, preferentially 18 through 1500 g/mol, especially preferentially 30 through 1000 g/mol.

The composition (Z1) preferably contains (further) one or more substances selected from the group (G2″)/(G3), consisting of water, methanol, ethanol, propanol, iso-propanol, butanol, pentanol, iso-amylalcohol, hexanol, heptanol, octanol, decanol, dodecanol, hexadecanol, octadecanol, propandiol, butandiol, pentandiol, hexandiol, heptandiol, octandiol, decandiol, dodecandiol, glycerol, sorbitol, propylene glycol, dipropylene glycol, triethyl citrate, methylcellulose, panthenol, iso-amylalcohol, methylbutanol, glucose, starch, formic acid, acetic acid, propionic acid, butyric acid, valeric acid, iso-valeric acid, caproic acid, caprylic acid, pelargonic acid, capric acid, myristic acid, stearic acid, lauric acid, palmitic acid, lactic acid, citric acid, oxalic acid, Na/K formate, Na/K acetate, Na/K propionate, Na/K butyrate, Na/K capronate, Na/K caprylate, Na/K decanoate, Na/K myristate, Na/K laurate, Na/K/Mg/Al/Zn stearate, Na/K palmitate, phytanoic acid, iso-propyl myristate, diethyl phthalate, methyl paraben, ethyl paraben, propyl paraben, lauryl sulfate, sorbitan tristearate, sorbitan monolaurate, sorbitan monooleate, sorbitan trioleate, sorbitan monopalmitate, lauryl ether sulfate, myristyle ether sulfate, paraffin, vasiline, propane, butane, iso-butane, pentane, methybutane, hexane, heptane, octane, nonane, decane, docane, tridecane, undecane, tetradecane, pentadecane, hexadecane, heptadecane, octadecane, menthan, cyclohexane, phytane, squalane, sterane, guaian, cedran, disodium dihydrogen diphosphate, tetra sodium diphosphate, dimethicone, cetylpyridinium chloride, polyquaternium, benzalkonium chloride, cetyltrimethylammonium bromide, benzalkonium chloride and hydrolysed keratin, wherein the substance or rather the or one, more or all substances of the group (G2) or (G2′) contained in the composition (Z1) is or rather are selected from the previous group (G2″) or wherein the composition in addition to the contained substance(s) of the group (G2) or (G2′) contains one or more substance(s) of the previous group (G3).

Substances of the group (G1), being the cause for undesirable off-notes frequently are present only in trace amounts and are measurable in the compositions themselves or in the headspace above according to a preferred embodiment of the present invention in a concentration of 0.001 ng/μL through 1 g/L or rather at 25° C. and atmospheric pressure.

The determination of the concentration of the substance(s) of the group (G1) can exemplarily take place by a gas chromatographic analysis (GC) or by a liquid chromatographic analysis (LC) after am extraction- or processing step. This determination of concentration is a routine analysis and is known to the person skilled in the art.

The determination of the concentration of the substance(s) of the group (G1) in the vapour phase above the compositions takes place preferably by a dynamic headspace sampling, wherein a certain volume of the headspace with substance(s) of the group (G1) is sucked off and the substance(s) is or rather are adsorbed on the carrier matrix. This loaded carrier matrix can by the action of heat (thermally) release the adsorbed substance(s) again in a GC (desorption) and thus the concentration of the adsorbed substances(s) can be determined in the volume of the headspace. Corresponding examples of analyses are described in detail in the Gerstel-applications AppNote-2011-01 and AppNote-2012-06. Another common method is the use of a solvent, which desorbs the adsorbed substance(s) and subsequently the concentration of the off-notes in the so gained solution can be determined analogously to the previously described approach.

A method for the qualitative determination of the off-note(s) is given by the common technique of the GC-olfactometry, which allows a sensory evaluation of the compounds separated by GC and thus the identification of the compounds relevant for smell and aroma. This is a common method, for which there is extensive technical literature (e.g. Delahuntry C M et al. 2006: Gas-Chromatography-olfactometry. J. Sep. Sci. 29 (14), 2107-25.)

The sensory examinations carried out in the scope of the present invention occurred via a sensorially-trained group of 15 persons, wherein the samples were randomized and coded, and both the perceived intensity and the perceived sensory impression itself was described. This group evaluated the intensity of the perceived impressions on a scale from 0 through 9, wherein 0 stands for no perceived intensity and 9 for a highly intense intensity impression. Furthermore, the sensorially perceived impressions were described with different attributes by the group.

The compositions as obtained in the scope of the present invention or rather resulting in the scope of the use according to the invention or a method according to the invention (as described herein) (cf. therefore particularly composition (Z2) as described herein further below) can be combined for the production of certain preparations (Z3), particularly aromatized and/or perfumed preparations (Z3), with further ingredients, additives and auxiliaries. Preferred preparations are thereby such from the fields of cosmetics, personal care, oral hygiene products, oral care, household, air fresheners, fine fragrance, perfumes, foodstuff, food supplements, flavours, pet food and pharmaceuticals.

The (further) ingredients mentioned above, auxiliaries or other additives are exemplarily surfactants, oils, emulsifiers, waxes, sugars, carbohydrates, proteins, egg whites, amino acids, sweeteners, flavour modulators, odour maskers, thickeners, stabilizers, polymers, fats, lecithin, phospholipids, UV filters, antioxidants, humectants, antitranspirants, antisperspants, anti-dandruff agents, swelling agents, film formers, buffers, insect repellents, self-tanning agents, depigmentation agents, solvents, preservatives, flavourings, perfumes, dyestuffs, plastics, elastomers, stabilisers, vitamins, fillers, copolymers, polysaccharides, protein hydrolysates, electrolytes, minerals, plant extracts, aqueous extracts, concentrates, essential oils and antibacterially, fungicidally and or sporadically active substances.

Such preparations (Z3) contain in a preferred embodiment next to a composition (Z2) described herein (as described herein further below) regularly one or more flavourings or rather perfumes.

Flavourings or rather perfumes in the sense of this invention are defined by e.g. “Riechstoffe”, in Steffen Arctander, in “Perfume and Flavor Chemicals”, self-publishing, Montclair, N. J. 1969; H. Surburg, J. Panten, in “Common Fragrance and Flavor materials”, 5th Edition, Wiley-VCH, Weinheim 2006 or European or American case law, such as exemplarily the provision (EG) Nr. 2232/96 of the European Parliament and of the Council and the positive list, which is defined in the Implementing Regulation (EU) Nr. 872/2012 of the Commission. Typical examples for flavourings or rather perfumes are exempoarily acetophenone, allylcapronate, alpha-ionone, beta-ionone, anisaldehyde, anisyl acetate, anisyl formiate, benzaldehyde, benzothiazol, benzyl acetate, benzylalcohol, benzylbenzoate, beta-ionone, butyl butyrate, butyl capronate, butyli-denphthalide, carvone, camphene, caryophyllene, cineol, cinnamyl acetate, citral, citronellol, citronellal, citronellyl acetate, cyclohexyl acetate, cymol, damascone, decalactone, dihydrocumarine, dimethylanthranilate, dimethylanthranilate, dodecalactone, ethoxyethyl acetate, ethyl butyric acid, ethyl butyrate, ethyl caprinate, ethyl capronate, ethyl crotonate, ethyl furaneol, ethyl guaiacol, ethyl iso-butyrate, ethyl iso-valerianate, ethyl lactate, ethyl methyl butyrate, ethyl propionate, eucalyptol, eugenol, ethyl heptylate, 4-(phydroxyphenyl)-2-butanone, gamma-decalactone, geraniol, geranyl acetate, geranyl acetate, grapefruit aldehyde, methyldihydrojasmonate (e.g. Hedion®), heliotropin, 2-heptanone, 3-heptanone, 4-heptanone, trans-2-heptenal, cis-4-heptenal, trans-2-hexenal, cis-3-hexenol, trans-2-hexenoic acid, trans-3-hexenoic acid, cis-2-hexenyl acetate, cis-3-hexenyl acetate, cis-3-hexenyl capronate, trans-2-hexenyl capronate, cis-3-hexenyl formate, cis-2-hexyl acetate, cis-3-hexyl acetate, trans-2-hexyl acetate, cis-3-hexyl formate, para-hydroxybenzyl acetone, iso-amyl alcohol, iso-amyl iso-valerianate, iso-butyl butyrate, iso-butyraldehyde, iso-eugenol methylether, iso-propyl methylthiazole, lauric acid, leavulinic acid, linalool, linalool oxide, linalyl acetate, menthol, menthofuran, methylanthranilate, methylbutanol, methylbutanoic acid, 2-methylbutylacetat, methyl capronate, methyl cinnamate, 5-methylfurfural, 3,2,2-methylcyclopentenolon, 6,5,2-methylheptenon, methyldihydrojasmonate, methyljasmonat, 2-methylmethylbutyrat, 2-methyl-2-pentenolic acid, methylthiobutyrate, 3,1-methylthiohexanol, 3-methylthiohexylacetat, nerol, neryl acetate, trans, trans-2,4-nonadienal, 2,4-nonadienol, 2,6-nonadienol, 2,4-nonadienol, nootkatone, delta octalactone, gamma octalactone, 2-octanol, 3-octanol, 1,3-octenol, 1-octyl acetate, 3-octyl acetate, palmitic acid, paraldehyde, phellandrene, pentandione, phenylethyl acetate, phenylethyl alcohol, phenylethyl alcohol, phenylethyl iso-valerianate, piperonal, propionaldehyde, propyl butyrate, pulegone, pulegol, sinensal, sulfurol, terpinene, terpineol, terpinolene, 8,3-thiomenthanone, 4,4,2-thiomethylpentanon, thymol, delta-undecalactone, gamma-undecalactone, valencene, valerianic acid, vanillin, acetoin, ethyl vanillin, ethyl vanillin iso-butyrate (=3-ethoxy-4-iso-butyryloxybenzaldehyde), 2,5-dimethyl-4-hydroxy-3(2H)-furanone and its derivatives (thereby preferably homofuraneol (=2-ethyl-4-hydroxy-5-methyl-3(2H)-furanone), homofuronol (=2-ethyl-5-methyl-4-hydroxy-3(2H)-furanone and 5-ethyl-2-methyl-4-hydroxy-3(2H)-furanon), maltol and maltol-derivatives (thereby preferably ethyl maltol), coumarin and coumarinderivatives, gamma-lactone (thereby preferably gamma-undecalactone, gammanonalactone, gamma-decalactone), delta-lactones (thereby preferably 4-methyldeltadecalactone, massoilactone, delta-decalactone, tuberolactone), methyl sorbate, divanillin, 4-hydroxy-2(or 5)-ethyl-5(or 2)-methyl-3(2H)furanone, 2-hydroxy-3-methyl-2-cyclopentenone, 3-hydroxy-4,5-dimethyl-2(5H)-furanone, acetic acid iso-amyl ester, butanoic acid ethyl ester, butanoic acid-n-butyl ester, butanoic acid iso-amyl ester, 3-methyl-butanoic acid ethyl ester, n-hexanoic acid ethyl ester, n-hexanoic acid allyl ester, n-hexanoic acid-n-butyl ester, n-octanoic acid ethyl ester, ethyl-3-methyl-3-phenylglycidate, ethyl-2-trans-4-cis-decadienoate, 4-(p-hydroxyphenyl)-2-butanone, 1,1-dimethoxy-2,2,5-trimethyl-4-hexane, 2,6-dimethyl-5-hepten-1-al and phenyl acetaldehyde, 2-methyl-3-(methylthio)furan, 2-methyl-3-furanthiol, bis(2-methyl-3-furyl)disulfide, furfuryl mercaptan, methional, 2-acetyl-2-thiazoline, 3-mercapto-2-pentanone, 2,5-dimethyl-3-furanthiol, 2,4,5-trimethylthiazol, 2-acetylthiazol, 2,4-dimethyl-5-ethylthiazol, 2-acetyl-1-pyrroline, 2-methyl-3-ethylpyrazine, 2-ethyl-3,5-dimethylpyrazine, 2-ethyl-3,6-dimethylpyrazine, 2,3-diethyl-5-methylpyrazine, 3-iso-propyl-2-methoxypyrazine, 3-iso-butyl-2-methoxypyrazine, 2-acetylpyrazine, 2-pentylpyridinr, (e,e)-2,4-decadienal, (e,e)-2,4-nonadienal, (e)-2-octenal, (e)-2-nonenal, 2-undecenal, 12-methyltridecanal, 1-penten-3-one, 4-hydroxy-2,5-dimethyl-3(2H)-furanone, guaiacol, 3-hydroxy-4,5-dimethyl-2(5H)-furanone, 3-hydroxy-4-methyl-5-ethyl-2(5H)-furanone, cinnamaldehyde, cinnamyl alcohol, methyl salicylate, iso-pulegol as well as (here not explicitly mentioned) stereo isomers, enantiomers, positional isomers, diastereomers, cis/trans-isomers or rather epimers of these substances.

Furthermore, one or more taste modulators can be contained. Taste modulators in the sense of the invention are flavouring substances, which can influence the taste directly, and are exemplarily selected from the group consisting of monosodium glutamate, free glutamic acid, nucleotides or their pharmaceutically acceptable salts, strombines, theogallines as described in JP 2007 110988 A, pyridinebetaine-compounds as described in EP 1291342 B1, glutamic acid glycosides as described in WO 2002 087361 A1, malic acid glycosides as described in WO 2006 003107 A1, glutathione derivatives as described in EP 0181421 or WO 2007 042273 A1), alkyl pyridines (preferably alkyl pyridines as described in WO 2009 122318 A1 and WO 2009 1223319 A1), in particular 2-hexyl-, 2-heptyl and 2-cctylpyridine, (2E,6Z)—N-cyclopropylnona-2,6-dienamide, (2E,6Z)-N-ethylnona-2,6-dienamide, N-[(2E)-3,7-dimethylocta-2,6-dienyl]cyclopropane carboxamide, M-[(2-methoxy-4-methyl-phenyl)methyl]-N-[2-(5-methyl-2-pyridyl)ethyl]-oxamide, N-[(2,4-dimethoxyphenyl)methyl]-N-[2-(2-pyridyl)ethyl]-oxamide, N-[(2-methoxy-4-methylphenyl)methyl]-N-[2-(2-pyridyl)ethyl]oxamide, N-(1-propylbutyl)-1,3-benzodioxole-5-carboxamide, 1-(2-hydroxy-4-isobutoxy-phenyl)-3-(2-pyridyl)propane-1-one and 1-(2-hydroxy-4-methoxy-phenyl)-3-(2-pyridyl)propane-1-one, cinnamic acid amides as described in EP 2,529,632-B1 or WO 2013 000,673), lactisoles, hesperitin according to EP 1909599 A1, phloretin according to EP 1972203 B1 or EP 1998636 B1, hydroxy flavans according to US 2010 292175 AA, 4-hydroxy chalcones according to EP 1972203 B1, extracts based on Hydrangea dulcis according to EP 2298084 A2, or Rubus ssp.; mixtures of whey proteins with lecithins, yeast extracts, plant hydrolysates, pulverised vegetable (e.g. onion powder, tomato powder), plant extracts (e.g. from lovage or from mushrooms such as Shiitake), seaweed and mineral salt mixtures, particularly mineral salt mixtures according to US 2009 214728 AA, rubemamin or rubescenamin according to EP 2,529,632-B1.

According to the invention, furthermore, a method for modifying, reducing or eliminating one or more off-note(s) is specified, comprising following steps:
(i) providing a composition (Z0), containing
   one or more substance(s) with off-note (s), preferably as defined above, wherein the substance or rather the or one, more or all substances is or rather are selected from the group (G1), (G1′), (G1″) or (G1′″) as defined above,
   one or more substance(s) selected from the group (G2), (G2′) or (G2″) as defined above,
(ii) providing one or more compound(s) of Formula (I) as defined above,
(iii) adding the compound or rather the compound(s) of Formula (I) from step (ii) to the composition (Z0) from step (i), to obtain a composition (Z1) as defined above,
(iiii) thermally treating the composition (Z1) obtained after adding the compound or rather of the compounds of Formula (I) for 0.1 hours through 168 days, preferentially for 0.5 through 96 hours, especially preferentially for 0.5 through 48 hours, with a temperature from 15 through 120° C., preferentially from 22 through 80° C., especially preferentially from 30 through 50° C., and chemically converting of the substance or rather of the substances of the group (G1), (G1′), (G1″) or (G1′″) and thereby modifying, reducing or eliminating the undesirable off-note(s) via the compound(s) of Formula (I), so that a composition (Z2) is obtained, at which in comparison with the compositions (Z0) and (Z1) the undesirable off-note(s) is or rather are modified, reduced or eliminated.

A composition (Z0) described herein does according to a preferred embodiment of the present invention not feature or at most feature a small total amount of fragrances and/or aromas, preferably a total amount of fragrances and/or aromas of less than 1.1 wt.-%, further preferentially of less than 0.1 wt.-%, relative to the total weight of the composition (Z0), wherein the substances of the group (G1) with its off-note or rather their off-notes are not to be added to the fragrances and/or aromas.

For preferred embodiments, e.g. relating to the preferentially contained substances or rather substances to be used or rather compounds and their amounts or rather concentrations, the above shall apply mutatis mutandis in connection with a use according to the invention.

Furthermore, the previously described method can comprise a further step (11111):
adding hydroxide ions to the composition (Z2) obtained after adding the compound(s) of Formula (I) and after the thermal treatment
and thermally treating the composition obtained therefrom for 0.1 through 48 hours, preferentially for 0.5 through 36 hours, particularly for 0.5 through 24 hours, with a temperature from 15 through 120° C., preferentially from 22 through 80° C., particularly preferentially from 30 through 50° C., and optionally adjusting or neutralising the pH-value.

The method can further be favoured or supported by the following further (optional) steps:
irradiating the composition (Z1) obtained after adding the compound(s) of Formula (I) in step (iii) or irradiating the composition (Z1) during the thermal treatment in step (iiii), or irradiating the composition (Z2), or irradiating the composition (Z2) present or rather obtained before, during or after the step (iiiii) described above, with UV-light for 0.5 through 96 hours, preferentially for 0.5 through 48 hours, especially preferentially for 0.5 through 24 hours.
adding iron and/or copper ions to the composition (Z2) obtained after adding the compound or rather the compounds of Formula (I) and after the thermal treatment, preferentially in the mass ratio of the total amount of compound(s) of Formula (I) to the total amount of iron and/or copper ions in the range from 20:1 through 1:5, especially preferentially in the range from 10:1 through 1:2, particularly preferentially in the range from 5:1 through 1:1.

The present invention also concerns a composition (Z2), producible by or produced by a method as described above, wherein the composition (Z2) contains a total amount of compound(s) of Formula (I) in the range from 0.05 wt.-% and less, preferably 0.00001 through 0.05 wt.-%. Also here, thereby holds correspondingly for preferred embodiments, e.g. relating to the preferentially obtained or rather utilizable substances or rather compounds the above in connection with a use according to the invention. Further preferred characteristics of such a composition also result from the previous statements. A such composition (Z2) is especially preferentially to be applied or rather contained in preparations (Z3) or rather products described above.

In the following, the present invention will be explained in more detail on the basis of selected examples. Thereby all quantities refer to the weight (wt.-%) unless stated otherwise.

### EXAMPLE 1—BODY GEL

Upon application of a body gel on the skin or rather upon applying crème, after a certain time, also a certain undesirable off-note was perceived next to the perfuming, which was described as “technical, fishy, musty”. The analysis of the formulation via different methods resulted in that the used polyquat (quaternary amino compounds), which releases trimethylamine, was the reason for this undesirable off-note.

### Production of the Body Gel:

| | | | Mix A | Mix B | Mix C | Mix D | Mix E |
|---|---|---|---|---|---|---|---|
| Phase | Ingredients | INCI | [wt.-%] | [wt.-%] | [wt.-%] | [wt.-%] | [wt.-%] |
| A | Water | Water (Aqua) | 84.50 | 84.45 | 84.45 | 96.10 | 96.05 |
| | Glycerol | Glycerol | 2.00 | 2.00 | 2.00 | 2.00 | 2.00 |
| B | Polyquaternium- | Polyquaternium- | 0.80 | 0.80 | 0.8 | 0.80 | 0.80 |
| | 37 | 37 | | | | | |
| | Percarbamide | Urea peroxide | 0 | 0.05 | 0.05 | 0 | 0.05 |
| C | Sodiumhydroxide | Sodiumhydroxide, | 0.50 | 0.50 | 0.50 | 0.50 | 0.50 |
| | solution | water | | | | | |
| | 10% | | | | | | |
| D | Hydrochloric | Hydrochloric | 0.60 | 0.60 | 0.60 | 0.60 | 0.60 |
| | acid solution | acid, water | | | | | |
| | 10% | | | | | | |
| E | Symdiol ®68 | 1,2- | 0.50 | 0.50 | 0.50 | 0 | 0 |
| | | hexandiol, | | | | | |
| | | caprylyl | | | | | |
| | | glycol | | | | | |
| | Hydrolite ® 5 | Pentylene | 5.00 | 5.00 | 5.00 | 0 | 0 |
| | | glycol | | | | | |
| | Extrapone ® | Water, | 1.00 | 1.00 | 1.00 | 0 | 0 |
| | Hibiscus | propylene | | | | | |
| | | glycol, glucose, | | | | | |
| | | Hibiscus | | | | | |
| | | extract | | | | | |
| | Timiron Sun | Mica, titanium | 0.10 | 0.10 | 0.10 | 0 | 0 |
| | gold Sparkle | dioxide, | | | | | |
| | MP-29 | iron oxide | | | | | |
| F | Neo-PCL water | Trideceth-9, | 3.00 | 3.00 | 3.00 | 0 | 0 |
| | Soluble N | PEG-5, ethyl | | | | | |
| | | hexanoate, | | | | | |
| | | water | | | | | |
| | Perfume | Perfume | 1.00 | 1.00 | 0.95 | 0 | 0 |
| | Propylene | Propylene | 0 | 0 | 0.05 | 0 | 0 |
| | glycol | glycol | | | | | |
| | Solubilizer | PEG-40 | 1.00 | 1.00 | 1.00 | 0 | 0 |
| | | Hydrogenated | | | | | |
| | | castor oil, | | | | | |
| | | tridedeth-9, | | | | | |
| | | propylene | | | | | |
| | | glycol, water | | | | | |

Phase A and phase B were mixed and dissolved homogeneously while stirring at 40° C. for 2 hours. After the addition of phase C, is was stirred further for 1 hour at 40° C. After that, phase D was added while stirring and the temperature was lowered to room temperature. After mixing phase E, it was added while stirring. Then, phase F was mixed and added to the batch while stirring.

### Application Test:

150 mg of each mixture was distributed evenly over a forearm skin area of 7×3 cm using a spatula. After about 30 seconds, the off-note was perceptible and was examined both sensorially and analytically. Therefore, the perfumed mixtures A, B and C as well as the unperfumed mixtures D and E were compared.

### Sensory Examination:

The sensory examinations were carried out by means of a sensory-trained group of 15 persons, whereby the samples were randomized and coded, whereby both the perceived intensity and the perceived sensory impression itself were described. This group evaluated the intensity of the perceived impressions (total intensity), as well as the perceived intensity of the off-note on a scale from 0 to 9, whereby 0 stands for no perceived intensity and 9 for a highly intense intensity impression. Furthermore, the group described the sensory impressions with different attributes.

a) Results of the Perfumed Mixtures A, B and C:

| | Perfumed mixtures | | |
|---|---|---|---|
| | Mix A | Mix B | Mix C |
| Total intensity | 6.3 | 7.2 | 6.4 |
| Intensity of the | 4.7 | 1.5 | 1.4 |
| off-note | | | |
| Description | flowery, fruity, | fresh, fruity, | flowery, fresh, |
| | fishy, technical, | flowery, musty | fruity, musty |
| | musty | | |

| | | |
|---|---|---|
| Total intensity in comparison with | Increase by 14% | comparable |
| Mix A | | |
| Intensity of the off-note in | Reduction by | Reduction by |
| comparison with Mix A | 68% | 70% |

The intensity of the off-note of mixture A was classified as perceptible (4) to clearly perceptible (5), while mixtures B and C were rated as barely perceptible (1) to weakly perceptible (2). Thus, mixtures B and C showed a significant reduction of the off-note intensity compared to mixture A.

b) Results of the Unperfumed Mixtures D and E:

Unperfumed mixtures

| | Mix D | Mix E |
|---|---|---|
| Total intensity | 5.1 | 1.5 |
| Intensity of the | 5.1 | 1.5 |
| off-note | | |
| Description | fishy, technical, | technical, musty |
| | musty | |

| | |
|---|---|
| Intensity of the off-note in | Reduction by |
| comparison with Mix D | 71% |

Since these were unperfumed mixtures, only the off-note was perceptible and the overall intensity was identical to the off-note intensity. The intensity of the off-note of mixture D was clearly perceived (5), while the mixture E was rated as barely perceptible (1) to weakly perceptible (2). Thus the mixture E showed a clear reduction of the off-note intensity of 71% compared to mixture D.

Analytical Examination:

The trimethylamine, the cause of the undesirable off-note, could only be analysed in the trace range at the detection limit of the GC-MS in the unperfumed mixtures D and E. In the perfumed mixtures A, B and C coelutions of the trimethylamine with other ingredients occurred. The determination of the trimethylamine was carried out in the vapour phase over the mixture applied to the forearm, analogously to the sensory evaluation. This headspace with the trimethylamine was continuously sucked off and the trimethylamine was deposited on the Tenax carrier matrix. The headspace sampling was performed at room temperature and at a flow of 100 mL/min for 15 minutes. This carrier matrix loaded with trimethylamine was sampled by means of a GC/MS and the MS areas (counts) were determined. From the determined areas of mixtures D and E, a factor was determined by proportioning the proportions.

Unperfumed
mixtures

| | Mix D | Mix E |
|---|---|---|
| Areas trimethylamine | 82586 | 7859 |

| | |
|---|---|
| Area factor von mixture D and E | 10.5 |

The area and thus the released amount of trimethylamine has been reduced by a factor of 10.5.

### EXAMPLE 2—ETHANOL

Ethanol is used, among others, for producing flavour blends for products in the food, cosmetics (personal care), household (home care), fine fragrance, oral care, pharmaceuticals and pet food sectors. Depending on the production and quality of the ethanol, it may contain undesirable off-notes, which are annoying in the product and reduce its quality. The undesirable off-note was described as “greasy, rancid, fermented, cheesy, spicy”. The investigations showed that this unwanted off-note was mainly caused by acrolein, butanal, hexanal, butanedione, acetoin and pentanedione.

Sample Production:

| | | I | II | III | IV | V | VI |
|---|---|---|---|---|---|---|---|
| Phase | Ingredient | [wt.-%] | [wt.-%] | [wt.-%] | [wt.-%] | [wt.-%] | [wt.-%] |
| A | Ethanol | 100 | 0 | 99.84 | 94 | 0 | 93.84 |
| | with off- | | | | | | |
| | note | | | | | | |
| B | Ethanol | 0 | 100 | 0 | 0 | 94 | 0 |
| | p.A. | | | | | | |
| C | Sodium | 0 | 0 | 0.05 | 0 | 0 | 0.05 |
| | percarbonate | | | | | | |
| D | Sodium | 0 | 0 | 0.05 | 0 | 0 | 0.05 |
| | hydroxide | | | | | | |
| | solution | | | | | | |
| | 10% | | | | | | |
| E | Hydrochloric | 0 | 0 | 0.06 | 0 | 0 | 0.06 |
| | acid | | | | | | |
| | solution | | | | | | |
| | 10% | | | | | | |
| F | Aroma | 0 | 0 | 0 | 6 | 6 | 6 |

To prepare the samples, the phase C was added to the phase A in a glass vessel and stirred closed for 24 hours at room temperature. Then, the phase D was added and stirred closed for 4 hours at room temperature. While stirring, the addition of the phase E was made, then the addition of the phase F was made.

Sensory Evaluation:

The samples were tempered to room temperature in a closed screw-cap glass vessel, wherein the same vessels and sample volumes were used. For each measurement, a maximum of 6 to 10 samples was evaluated, and blank samples with tap water as well as some double identical samples were measured. The sensory evaluation was performed by a sensory-trained group of 15 persons as well as randomized and coded samples, wherein both the perceived intensity and the perceived sensory impression itself were described. This group evaluated the intensity of the perceived impressions (total intensity) as well as the perceived intensity of the off-note on a scale from 0 to 9, wherein 0 stands for no perceived intensity and 9 for a highly intense intensity impression. Furthermore, the sensory impressions were described with different attributes by the group.

| | Total | Intensity | | |
|---|---|---|---|---|
| | intensity | off-note | Description | Results |
| Blind | 0.1 | 0 | neutral, odourless | Comparison of the off- |
| I | 4.8 | 4.5 | greasy, fermented, cheesy, | note intensity of the |
| | | | vegetables, rancid, alcoholic, | initial sample I with the |
| | | | spicy | treated sample III |
| II | 1.5 | 0.4 | alcoholic, neutral, technical, | Reduction by 90% |
| | | | medicinal | |
| III | 1.8 | 0.5 | alcoholic, greasy, spicy | Comparison of the off- |
| IV | 6.0 | 4.4 | greasy, vegetables, spicy, | note intensity of the |
| | | | orange, citrus, fruity, flowery | initial sample IV with |
| V | 6.3 | 0 | citrus, orange, fresh, fruity, | the treated sample VI |
| | | | flowery | Reduction by 91% |
| VI | 6.1 | 0.4 | citrus, orange, fruity, flowery, | |
| | | | spicy | |

The treated sample III showed a reduction of the off-note intensity by 90% and tended to be comparable with sample II (ethanol p.A.). The same tendencies were found in the aromatized samples. In the treated aromatized sample VI, the intensity of the off-note was evaluated as barely perceptible and was significantly reduced by 91%.p

Analytical Examination:

The unwanted off-note was mainly caused by acrolein, butanal, hexanal, butanedione, acetoin and pentanedione and could only be analyzed in the trace range by GC-MS in the non-aromatized samples I and III. In the aroma containing samples IV and VI, the substances causing the off-notes were masked by the added aroma substances and could therefore not be analyzed. The determination of the individual off-note components was carried out, analogous to the sensory evaluation, in the vapour phase above the sample in the screw cap jars at room temperature. This headspace with the off-note components was continuously sucked off, whereby the off-note components then accumulated on the Tenax carrier matrix. The headspace sampling was performed at room temperature and a flow of 100 mL/min for 30 minutes. The carrier matrix loaded with the off-note components was measured using GC/MS and the MS areas (counts) of the individual compounds causing the off-notes were evaluated. For the determination of the total off-note, the areas and thus the quantities of the individual off-note components were combined to a total area. These total areas of the samples I and III were put into relation and a factor was determined.

| | Sample I | Sample III |
|---|---|---|
| Total surface area off-note | 5252214 | 535741 |
| components | | |

| | |
|---|---|
| Area factor of mixture D and E | 9.8 |

The total surface area of the off-note components and thus the quantity in the headspace has been reduced by a factor of 9.8.

1-14. (canceled)

15. A method for modifying, reducing, or eliminating one or more off-notes of one or more substances of (G1), the method comprising combining one or more compounds of Formula (I) with one or more substances of (G1) in a composition (Z1):
the compounds of Formula (I) represented by:
XₖYₗZₘOₙ (I)
wherein X=N, Na, K, Mg or Ca,
k=0, 1 or 2,
Y=C or S,
1=0, 1, 2 or 3,
Z=H, with m=0, 1, 2, 3, 4, 5, 6, 7 or 8, and
n=2, 3, 4, 5, 6, 7 or 8; and
the composition (Z1) comprises:
one or more compounds of Formula (I),
(G1) one or more substances selected from aldehydes, ketones, unsaturated hydrocarbons, and amines, and
(G2) one or more substances selected from alcohols, carboxylic acids, esters, carboxylates, mineral salts, saturated hydrocarbons, silicones, quaternary ammonium compounds, sulfates, and sulphuric acid esters.

16. The method of claim 15, wherein composition (Z1) comprises 0.001 to 30 wt. % of the one or more compounds of Formula (I), based on the total weight of composition (Z1).

17. The method of claim 15, wherein composition (Z1) comprises up to 0.1 wt. % of the one or more substances of (G1), based on the total weight of composition (Z1).

18. The method of claim 15, wherein the mass ratio of the total amount of compounds of Formula (I) to the total amount of substances of group (G1) in composition (Z1) 300:1 to 1:3.

19. The method of claim 15, wherein the molecular weight of the one or more compounds of Formula (I) is 500 g/mol to 30 g/mol.

20. The method of claim 15, wherein the one or more off-notes are selected from roasted, nutty, spicy, bitter, stinging, biting, scratchy, medicinal, fishy, aminous, hay-like, chemical, technical, burnt, fatty, sour, herbaceous, musty, rotten, fermented, terpenous, aromatic, fruity, woody, fleshy, ethereal, earthy, green, solvent-like, rubbery, charred, faecal, urinary, earthy, mushroomy, smoky, decaying, rancid, gasoline-like, buttery, vegetable-like, cooked, animalistic, sweaty, cheesy, cabbage-like, hot, cooling, creamy, flowery, ointment-like, mossy, metallic, workshop-like, indoor swimming pool-like, pyridine-like, pyrazine-like, ensilage-like, carrot-like, adhesive-like, disinfectant-like, dusty, spicy, powdery, astringent, papery, dusty, dry, and floury off-notes.

21. The method of claim 15, wherein the one or more substances of (G1) are selected from the substances of (G1′):
(G1′) linear, branched, cyclic or aromatic aldehydes; linear, branched, cyclic or aromatic ketones; and branched or cyclic amines

22. The method of claim 15, wherein the one or more substances of G2) are selected from the substances of (G2′):
(G2′) compounds with a molecular mass in the range of 18 through 2000 g/mol.

23. The method of claim 15, wherein composition (Z1) further comprises:
(G3) one more substances selected from water, methanol, ethanol, propanol, iso-propanol, butanol, pentanol, iso-amylalcohol, hexanol, heptanol, octanol, decanol, dodecanol, hexadecanol, octadecanol, propandiol, butandiol, pentandiol, hexandiol, heptandiol, octandiol, decandiol, dodecandiol, glycerol, sorbitol, propylene glycol, dipropylene glycol, triethyl citrate, methylcellulose, panthenol, iso-amylalcohol, methylbutanol, glucose, starch, formic acid, acetic acid, propionic acid, butyric acid, valeric acid, iso-valeric acid, caproic acid, caprylic acid, pelargonic acid, capric acid, myristic acid, stearic acid, lauric acid, palmitic acid, lactic acid, citric acid, oxalic acid, Na/K formate, Na/K acetate, Na/K propionate, Na/K butyrate, Na/K capronate, Na/K caprylate, Na/K decanoate, Na/K myristate, Na/K laurate, Na/K/Mg/Al/Zn stearate, Na/K palmitate, phytanoic acid, iso-propyl myristate, diethyl phthalate, methyl paraben, ethyl paraben, propyl paraben, lauryl sulfate, sorbitan tristearate, sorbitan monolaurate, sorbitan monooleate, sorbitan trioleate, sorbitan monopalmitate, lauryl ether sulfate, myristyle ether sulfate, paraffin, vasiline, propane, butane, iso-butane, pentane, methybutane, hexane, heptane, octane, nonane, decane, docane, tridecane, undecane, tetradecane, pentadecane, hexadecane, heptadecane, octadecane, menthan, cyclohexane, phytane, squalane, sterane, guaian, cedran, disodium dihydrogen diphosphate, tetra sodium diphosphate, dimethicone, cetylpyridinium chloride, polyquaternium, benzalkonium chloride, cetyltrimethylammonium bromide, benzalkonium chloride and hydrolysed keratin.

24. A method for modifying, reducing or eliminating one or more off-notes comprising:
(i) providing a composition (Z0) comprising:
one or more substances with off-notes; and
one or more substances selected from (G2), (G2′), and (G2″):
(G2) alcohols, carboxylic acids, esters, carboxylates, mineral salts, saturated hydrocarbons, silicones, quaternary ammonium compounds, sulfates and sulphuric acid esters;
(G2′) compounds with a molecular mass in the range from 18 through 2000 g/mol; and
(G2″) water, methanol, ethanol, propanol, iso-propanol, butanol, pentanol, iso-amylalcohol, hexanol, heptanol, octanol, decanol, dodecanol, hexadecanol, octadecanol, propandiol, butandiol, pentandiol, hexandiol, heptandiol, octandiol, decandiol, dodecandiol, glycerol, sorbitol, propylene glycol, dipropylene glycol, triethyl citrate, methylcellulose, panthenol, iso-amylalcohol, methylbutanol, glucose, starch, formic acid, acetic acid, propionic acid, butyric acid, valeric acid, iso-valeric acid, caproic acid, caprylic acid, pelargonic acid, capric acid, myristic acid, stearic acid, lauric acid, palmitic acid, lactic acid, citric acid, oxalic acid, Na/K formate, Na/K acetate, Na/K propionate, Na/K butyrate, Na/K capronate, Na/K caprylate, Na/K decanoate, Na/K myristate, Na/K laurate, Na/K/Mg/Al/Zn stearate, Na/K palmitate, phytanoic acid, iso-propyl myristate, diethyl phthalate, methyl paraben, ethyl paraben, propyl paraben, lauryl sulfate, sorbitan tristearate, sorbitan monolaurate, sorbitan monooleate, sorbitan trioleate, sorbitan monopalmitate, lauryl ether sulfate, myristyle ether sulfate, paraffin, vasiline, propane, butane, iso-butane, pentane, methybutane, hexane, heptane, octane, nonane, decane, docane, tridecane, undecane, tetradecane, pentadecane, hexadecane, heptadecane, octadecane, menthan, cyclohexane, phytane, squalane, sterane, guaian, cedran, disodium dihydrogen diphosphate, tetra sodium diphosphate, dimethicone, cetylpyridinium chloride, polyquaternium, benzalkonium chloride, cetyltrimethylammonium bromide, benzalkonium chloride and hydrolysed keratin;
(ii) providing one or more compounds of the Formula (I),
XₖYₗZₘOₙ (I),
wherein X=N, Na, K, Mg or Ca,
k=0, 1 or 2,
Y=C or S,
1=0, 1,2 or 3,
Z=H,
m=0, 1, 2, 3, 4, 5, 6, 7 or 8, and
n=2, 3, 4, 5, 6, 7 or 8,
(iii) adding the one or more compounds of Formula (I) to composition (Z0) to obtain a composition (Z1),
(iv) thermally treating composition (Z1) for 0.1 hours through 168 days at a temperature of 15 to 120° C. and chemically converting the one or more substance selected from (G2), (G2′), and (G2″) to modify, reduce, or eliminate the undesirable off-notes via the compounds of the Formula (I), so that a composition (Z2) is obtained such that in comparison with the compositions (Z0) and (Z1) the undesirable off-notes are modified, reduced, or eliminated.

25. The method of claim 24, further comprising:
(v) adding hydroxide ions to composition (Z2) after the thermal treatment and thermally treating the composition obtained therefrom for 0.1 through 48 hours.

26. The method of claim 24, further comprising irradiating composition (Z1) with UV-light for 0.5 to 96 hours.

27. The method of claim 24, further comprising adding iron and/or copper ions to composition (Z2).

28. A composition (Z2) producible by the method of claim 24, wherein composition (Z2) comprises up to 0.05 wt. % of the one or more compound of Formula (I), based on the total weight of composition (Z2).

29. The method of claim 15, wherein the composition (Z1) comprises 0.01 through 20 wt. % of the one or more compounds of Formula (I), based on the total weight of composition (Z1).

30. The method of claim 15, wherein the composition (Z1) comprises up to 0.001 wt. % of the one or more substances of (G1), based on the total weight of composition (Z1).

31. The method of claim 15, wherein the mass ratio of the total amount of compounds of Formula (I) to the total amount of substances of group (G1) in composition (Z1) from 10:1 through 1:1.

32. The method of claim 15, wherein the molecular weight of the one or more compounds of Formula (I) is 160 g/mol to 30 g/mol.

33. The method of claim 15, wherein the one or more substances of (G1) are selected from the substances of (G1″):
(G1″) compounds with a molecular mass of 18 to 700 g/mol.

34. The method of claim 15, wherein the one or more substances of (G1) are selected from the substances of (G1′″):
(G1′″) formaldehyde, acetaldehyde, propanal, butanal, pentanal, hexanal, heptanal, octanal, nonanal, decanal, undecanal, dodecanal, tetradecanal, hexadecanal, octadecanal, decadienal, dodecandienal, heptadienal, hexadienal, octadienal, undecandienal, decenal, undecenal, heptenal, trienale and further, octenone, octenal, nonenal, decenal, propanone, butanone, pentanone, pentenone, octadienone, methylnonadienone, pentylfuran, hexanone, heptanone, decanone, methylheptenone, methylheptadienone, methylpropanal, methylbutanal, phenylethanal, hydroxymethylfuranone, hydroxydimethylfuranone, hydroxyethylfuranone, acrolein, benzaldehyde, furfural, furylidenketone, carene, indene, squalene, ocimene, limonene, terpinene, phellandrene, pinene, myrcene, tridecene, tetradecene, dodecene, pentadecene, undecene, decene, nonene, octadecene, heptadecene, hexadecene, farnesene, humulene, butene, pentene, methylpentene, butandiene, methylbutadiene, undectriene, longifolen, menthadiene, methylbutene, styrene, methylamine, dimethylamine, trimethylamine, ethylamine, diethylamine, triethylamine, butylamine, ethanolamine, diethanolamine, triethanolamine and furfurylamine.
